(19) United States
(12) Patent Application Publication
(10) Pub. No.: US 2003/0049317 A1
(43) Pub. Date: Mar. 13, 2003

(54) Method and composition for reducing the danger and preventing the abuse of controlled release pharmaceutical formulations

(76) Inventor: David R. Lindsay, Durham, NC (US)

Correspondence Address: OLIVE & OLIVE, P.A., 500 MEMORIAL STREET, PO BOX 2049, DURHAM, NC, 27702, US

(21) Appl. No.: 09/943,072

(22) Filed: Aug. 30, 2001

(51) Int. Cl. A61K 9/22; A61K 31/485

(52) U.S. Cl. 424/468; 514/282

(57) ABSTRACT

The present invention relates to a method and composition for reducing the danger and preventing the abuse of orally administered, controlled release, pharmaceutical formulations. These formulations are useful to prevent the abuse of or accidental overdose of such formulations which can be overdosed or abused by chewing or crushing the formulation prior to ingestion. The formulations comprise a therapeutic, pharmaceutical composition and an antagonist to the pharmaceutical composition which is only bioavailable upon chewing or crushing of the antagonist prior to oral administration of the pharmaceutical formulation.

### FIELD OF THE INVENTION

This invention relates to pharmaceutical formulations in controlled release dosage form that have the potential for abuse or harm to a patient if the entire amount of the composition is made bioavailable at one time. The invention specifically relates to a controlled release pharmaceutical formulation which contains a therapeutic amount of a pharmaceutical composition in a controlled release dosage form and an antagonist to the pharmaceutical composition wherein the antagonist is in a form which is only made bioavailable orally upon chewing or crushing of the dosage form of the antagonist prior to oral administration. This invention particularly relates to pharmaceutical formulations of controlled substances and especially opioids.

### BACKGROUND OF THE INVENTION

Pharmaceutical formulations are mixtures, matrixes, blends, etc. of ingredients, both pharmacologically active and inactive, intended to deliver medications for the treatment of a particular condition of a human or animal. Typically, pharmaceutical formulations contain a pharmaceutical composition along with fillers, binders, excipients, and the like in a form designed to efficiently maximize the administration and desired blood levels of the active pharmaceutical composition. The route of administration of concern in this invention is oral. Pharmaceutical compositions are bioactive chemicals, drugs, natural products, and the like intended for administration to a human or animal for the purpose of eliciting a particular pharmaceutical effect, normally therapeutic in nature.

In an attempt to provide convenience to patients needing to be treated with oral pharmaceutical formulations, controlled release pharmaceutical formations have been developed, in both tablet and capsule form, which allow up to a twenty-four hour dosage of a pharmaceutical composition to be delivered that would otherwise need to be administered orally every four hours or so. A problem exists in that these pharmaceutical formulations, both capsules and tablets, will deliver the entire active dosage, i.e. make it bioavailable, to the patient all at once if the tablet or contents of the capsule is chewed or crushed prior to oral administration. In the case of a pharmaceutical composition designed for the treatment of a medical condition, it could result in a toxic overdose. In the case of a controlled substance, chewing or crushing of the pharmaceutical formulation before oral administration could be a method for abusing the controlled substance. Numerous techniques exist for preparing controlled release pharmaceutical formulations. One common technique involves surrounding an osmotically active pharmaceutical composition core with a semipermeable membrane. The pharmaceutical composition is released from the membrane over time by allowing a fluid, such as gastric or intestinal fluid, to permeate the coating membrane and dissolve the pharmaceutical composition so the dissolved composition can permeate the membrane. In some cases a hydrogel is employed to push the active ingredient through the membrane.

Another common technique for preparing controlled release pharmaceutical formulations is to encapsulate a plurality of pills, pellets or granules containing a pharmaceutical composition with varying levels of a diffusion barrier and/or different types of the diffusion barriers and encasing the result in a hard gelatin capsule.

Controlled substances are specific pharmaceutical compositions which have been classified by federal law as having the potential for abuse and as such their distribution is heavily regulated. They include both illegal compositions, such as mescaline and LSD, as well as therapeutic compositions such as analgesics, central nervous system stimulants, and some of the major and minor tranquilizers. Especially noteworthy, because of the extremely high abuse potential, are the very strong analgesics, the opioids.

Opioids, also known as opioid agonists, are a group of pharmaceutical compositions that exhibit opium or morphine-like properties. The opioids are employed primarily as moderate to strong analgesics, but have many other pharmacological effects as well, including drowsiness, respiratory depression, changes in mood and mental clouding without a resulting loss of consciousness. Opioids act as agonists, interacting with stereo specific and saturable binding sites in the brain and other tissues. Endogenous opioid-like peptides are present particularly in areas of the central nervous system that are presumed to be related to the perception of pain; to movement, mood and behavior, and to the regulation of neuroendocrinological functions. Opium contains more than twenty distinct alkaloids.

The chemical classes of opioids with morphine-like activity are the purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives.

The principal phenanthrenes are morphine, codeine, and thebaine. The principal benzoisoquinolines are papaverine, a smooth muscle relaxant, and noscapine. Semi-synthetic derivatives of morphine include diacetylmorphine (heroin), hydromorphone, oxymorphone, hydrocodone, apomorphine, etorpine, and oxycodone. Phenylpiperidine derivatives include meperidine and its congeners diphenoxylate and loperamide, alphaprodine, anileridine hydrochloride or phosphate, and piminodine esylate. Morphinan derivatives include levorphanol. The diphenyl-heptane derivatives include methadone and its congeners, and propoxyphene. Propionanilide derivatives include fentanyl citrate and its congeners sufentanil citrate and alfentanil hydrochloride.

By the middle of the nineteenth century, the use of pure alkaloids such as morphine rather than crude opium preparations began to spread throughout the medical world. Parenteral use of morphine tended to produce a more severe variety of compulsive drug use. The problem of addiction to opioids stimulated a search for potent analgesics that would be free of the potential to produce addiction. By 1967, researchers had concluded that the complex interactions among morphine-like drugs, antagonists, and what was then called “mixed agonist-antagonist” could best be explained by postulating the existence of more than one type of receptor for opioids and related drugs. With the advent of new totally synthetic entities with morphine-like actions, the term “opioid” was generally retained as a generic designation for all exogenous substances that bind stereo-specifically to any of several subspecies of opioid receptors and produce agonist actions.

The potential for the development of tolerance and physical dependence with repeated opioid use is a characteristic feature of all the opioid drugs, and the possibility of developing psychological dependence (i.e., addiction) is one of the major concerns of pain treatment with opioids, even though iatrogenic addiction is rare.

The overall abuse potential of an opioid is not established by any one single factor. Instead, there is a composite of factors, including; the capacity of the drug to produce the kind of physical dependence in which drug withdrawal causes sufficient distress to bring about drug-seeking behavior; the ability to suppress withdrawal symptoms caused by withdrawal from other agents; the degree to which it induces euphoria similar to that produced by morphine and other opioids; the patterns of toxicity that occur when the drug is dosed above its normal therapeutic range; and physical characteristics of the drugs such as water solubility. Such physical characteristics may determine whether the drug is likely to be abused.

In the United States, the effort to control the compulsive drug user includes efforts to control drug availability by placing restrictions on the use of opioids in the treatment of pain of compulsive drug users. In practice, the physician is often faced with a choice of administering potent opioid analgesics even to persons who seem predisposed to develop psychological dependence, i.e., addiction, on such drugs. In view of this problem, it has been recommended that these patients should not be given an opioid when another drug without a potential for abuse will suffice; and further that these patients should not be permitted to self-administer such drugs parenterally and they should only be given a few days' supply at a time.

At least three basic patterns of opioid use and dependence have been identified.

The first involves individuals whose drug use begins in the context of medical treatment and who obtain their initial supplies through physicians. Another pattern begins with experimental or “recreational” drug use and progresses to more intensive use. A third pattern involves users who begin in one or another of the preceding ways but later switches to oral opioids such as methadone, obtained from organized addiction treatment programs.

Tolerance refers to the need to increase the dose of opioid over a period of time in order to achieve the same level of analgesia or euphoria, or the observation that repeated administration of the same dose results in decreased analgesia, euphoria, or other opioid effects. It has been found that a remarkable degree of tolerance develops to the respiratory depressant, analgesic, sedative, emetic and euphorigenic effects of opioids. However, the rate at which this tolerance may develop in either an addict or in a patient requiring treatment of pain, depends on the pattern of use. If the opioid is used frequently, it may be necessary to increase the dose. Tolerance does not develop equally or at the same rate to all the effects of opioids, and even users who are highly tolerant to respiratory depressant effects continue to exhibit miosis and constipation. Tolerance to opioids largely disappears when the withdrawal syndrome has been completed.

Physical dependence may develop upon repeated administrations or extended use of opioids. Physical dependence is gradually manifested after stopping opioid use or is precipitously manifested (e.g., within 20 minutes) after administration of a narcotic antagonist (referred to as “precipitated withdrawal”). Depending upon the drug to which dependence has been established and the duration of use and dose, symptoms of withdrawal vary in number, kind, duration, and severity. The most common symptoms of the withdrawal syndrome include anorexia, weight loss, papillary dilation, chills alternating with excessive sweating, abdominal cramps, nausea, vomiting, muscle spasms, hyperirritability, lachrymation, rinorrhea, goose flesh and increased heart rate.

Abstinence syndrome typically begins to occur 24-48 hours after the last dose, and the syndrome reaches its maximum intensity about the third day and may not begin to decrease until the third week.

Psychological dependence (i.e., addiction) on opioids is characterized by drug-seeking behavior directed toward achieving euphoria and escape from, e.g., psychosocioeconomic pressures. An addict will continue to administer opioids for non-medicinal purposes and even in the face of self-harm.

Pharmacologically, antagonists typically block, neutralize, or reverse all of the effects of the pharmaceutical composition. One use of opioid antagonists is as a once-a-day treatment of naltrexone to block euphoric effects that might be otherwise obtained upon administration of opioids to addicts. Small doses of opioid antagonists have been used to determine whether individuals are physically dependent on opioids. Most commonly, opioid antagonists are used to reverse the effects of opoids on individuals who have overdosed on opioid agonist drugs.

There have been previous attempts in the medical field to control the abuse potential associated with opioid analgesics. Typically, a particular dose of an opioid analgesic is more potent when administered parenterally as compared to the same dose administered orally. Therefore, one popular mode of abuse of oral medications involves the extraction of the opioid from the oral dosage form, and the subsequent injection of the opioid (using any “suitable” vehicle for injection) in order to achieve a “high”. Attempts to curtail abuse have therefore typically centered around the inclusion, in the oral dosage form, of an opioid antagonist which is not orally active but which will substantially block the analgesic effects of the opioid if one attempts to dissolve the opioid and administer it parenterally.

For example, the combination of pentazocine and naloxone has been utilized in tablets available in the United States, commercially available as Talwin.RTM.Nx from Sanofi-Winthrop. Talwin.RTM.Nx contains pentazocine hydrochloride equivalent to 50 mg base and naloxone hydrochloride equivalent to 0.5 mg base. Talwin.RTM.Nx is indicated for the relief of moderate to severe pain. The amount of naloxone present in this combination has no action when taken orally, and will not interfere with the pharmacologic action of pentazocine. However, this amount of naloxone given by injection has profound antagonistic action to narcotic analgesics. Thus, the inclusion of naloxone is intended to curb a form of misuse of oral pentazocine which occurs when the dosage form is solubilized and injected. This dosage has lower potential for parenteral misuse than previous oral pentazocine formulations. However, it is still subject to patient misuse and abuse by the oral route, for example, by the patient taking multiple doses at once.

Other attempts have been previously made to use antagonists to control the abuse or overdose potential of pharmaceutical formulations, most notably with the opioid analgesics.

U.S. Pat. No. 3,773,955 (Pachter et al.) describes orally effective analgesic compositions which upon parenteral administration do not produce analgesia, euphoria, or physical dependence, and thereby prevent parenteral abuse of the analgesic agents. Such compositions contained from about 0.1 mg to about 10 mg naloxone per analgesic oral dose. This reference was not concerned with oral abuse of opioids.

U.S. Pat. No. 4,457,933 (Gordon et al.) describes a method for decreasing both the oral and parenteral abuse potential of strong analgesic agents such as oxycodone, propoxyphene and pentazocine, by combining an analgesic dose of the opioid with naloxone in a specific, relatively narrow range. Oxycodone-naloxone compositions having a ratio of 2.5-5:1 parts by weight and pentazocine-naloxone compositions having a ratio of 16-50:1 parts by weight were preferred. The dose of naloxone which was to be combined with the opioid is stated to substantially eliminate the possibility of either oral or parenteral abuse of the opioid without substantially affecting the oral analgesic activity thereof.

U.S. Pat. No. 6,228,863 (Palermo et al.) describes a method of reducing the abuse potential of an oral dosage form of an opioid analgesic wherein an analgesically effective amount of an orally active opioid agonist is combined with an opioid antagonist into an oral dosage form which would require at least a two-step extraction process to be separated from the opioid agonist, the amount of opioid antagonist included being sufficient to counteract the opioid effects if extracted together with the opioid agonist and administered parenterally.

Pharmaceutical formulations are now readily and often preferably available in controlled release dosage form. Controlled release formulations for oral administration are normally administered in the form of tablets or capsules. Tablets are normally formulated as a controlled release dosage of particles, granules seeds, matrices, coatings and the like and pressed into tablet form. Controlled release capsules consist of a hard gelatin capsule filled with pills, granules (often spherical) or pellets which releases medication at varying intervals. These dosage forms have the advantage of lowering the frequency of administration of the dosage to every 8, 12 or even 24 hours as opposed to the normal 4-6 hours for formulations which are not controlled release. See for example U.S. Pat. Nos. 4,990,341 and 4,844,909 (Goldie et al.) and U.S. Pat. No. 6,103,261 (Chasin et al.).

A problem with controlled release dosages of pharmaceutical formulations, however, is while they are more convenient for medical staff to administer, they can have a greater abuse potential and are potentially more dangerous than the non-controlled release time dosage forms. Specifically, if someone wants to get the entire dosage at once, either on purpose or accidentally, all that is necessary is to chew or crush the tablet or capsule contents prior to oral administration of the pharmaceutical formulation.

It would, therefore, be valuable and desirable to have a controlled release pharmaceutical formulation or method which reduces the abuse potential or danger of controlled release oral dosage forms if they are chewed or crushed prior to oral administration.

### SUMMARY OF THE INVENTION

It is an object of the present invention to provide a pharmaceutical formulation in a controlled release oral dosage form, especially one containing a controlled substance or an opioid analgesic. This formulation reduces the abuse potential and overdose danger to a patient if the pharmaceutical formulation is crushed or chewed prior to administration via the oral route. Prior commercially available controlled released oral dosage forms of pharmaceutical formulations have not met this need.

It is another object of the present invention to provide a method of reducing the abuse potential of and danger of a controlled release oral dosage form of a pharmaceutical formulation that may be crushed or chewed prior to oral administration. The present invention method combines a therapeutic amount of a controlled release form of a pharmaceutical composition with an antagonist to the pharmaceutical composition in sufficient quantity to counteract the effects of the pharmaceutical composition. The antagonist is provided in a dosage form that is only orally bioactive as an antagonist when the pharmaceutical formulation is crushed or chewed prior to oral administration.

It is another object of the present invention to provide a method of reducing the abuse potential or overdose danger of a controlled release oral dosage form of pharmaceutical formulation such that it has less potential for abuse, overdose, or misuse.

One aspect of the present invention, therefore, is directed to a controlled release, storage stable, pharmaceutical formulation intended for oral administration. The formulation comprises an orally active therapeutic amount of a pharmaceutical composition in a controlled release dosage form and an antagonist to the pharmaceutical composition in sufficient quantity to counteract the effects of the pharmaceutical composition when taken orally. The formulation is in a dosage form wherein the antagonist is only orally effective as an antagonist if it is chewed or crushed before oral administration.

### DETAILED DESCRIPTION OF THE INVENTION AND SPECIFIC EMBODIMENTS

The term pharmaceutical formulation as used in this specification refers to any pharmacologically active pharmaceutical composition, such as a therapeutic composition, medication, food supplement or the like in combination with inactive fillers, benders, excipients or the like, which can be or is given in controlled release dosage form and has the potential of abuse or overdose should the entire dosage become bioavailable at one time. It is anticipated that this bioavailability could occur upon either the purposeful or accidental chewing or crushing of the pharmaceutical formulation prior to oral administration. Such grinding or chewing would defeat the controlled release nature of the formulation and make the entire dosage of pharmaceutical composition bioavailable at once. Chewing or crushing its controlled release formulation can amount to the patient receiving higher than the intended dosage of pharmaceutical composition over a period of time intended for a single dosage. The potential for abuse, overdose, or even death, is obvious.

Antagonists, as used herein, are those orally active compositions which when present in sufficient dosage will block, neutralize, or reverse the effect of the pharmaceutical composition on the patient. These compositions are almost always orally active salts. It could include some compositions considered partial antagonists as well. It is also true that the amount of antagonist needed to counteract a pharmaceutical composition is much higher when considering oral routes of administration than would be the case where the pharmaceutical composition and an antagonist is administered intravenously.

Examples of prescription drugs would especially include the oral analgesics such as the opioids which have a tremendous potential for abuse. Examples of opioids currently available in a timed-release formulation include oxycodone (e.g. Oxycotin, Purdue) and morphine (e.g. Oramorph, Roxane; MS Contin, Purdue; Kadian, Faulding).

While the present invention is directed to any sustained release pharmaceutical composition, and an antagonist to the pharmaceutical composition, the examples are of the opioid/antagonist composition but the disclosure is intended to cover the broader invention of controlled release pharmaceutical formulations comprising a pharmaceutical composition with an antagonist which is only orally bioavailable if the antagonist is chewed or crushed prior to oral administration.

Opioid analgesics which are useful in the present invention include all opioids, agonists or mixed agonist-antagonists, partial agonists, including but not limited to alfentanil, allylprodince, alphaprodine, anileridine, benzylorphine, bezitrarnide, buprenorphine, butorphanol, clonirazene, codeine, desomorphine, dextromoramide, dezocine, diampromide, diamorphone, dihydrocodeine, dihydromorphine, dimenoxaldol, dimepheptanol, dimethylthiambutene, dioxaphetyl butyrate, dipipanone, eptazocine, ethopheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, matazocine, methadone, methpon, morphine, myrophine, narceine, micomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, mixtures of any of the foregoing, salts of any of the foregoing, and the like.

In certain embodiments, the opioid agonist or analgesic is selected from the group consisting of hydrocodone, morphine, hydromorphone, oxycodone, codeine, levorphanol, meperidine, methadone, or salts thereof, or mixtures thereof. In certain other embodiments, the opioid is hydrocodone. Equianalgesic doses of these opioids, in comparison to a 15 mg dose of hydrocodone, are set forth in Table 1 below.

TABLE 1
Equianalgesic doses of Opioids

| Opioid | Calculated Dose (mg) |
|---|---|
| Oxycodone | 13.5 |
| Codeine | 90.0 |
| Hydrocodone | 15.0 |
| Hydromorphone | 3.375 |
| Levorphanol | 1.8 |
| Meperidine | 135.0 |
| Methadone | 9.0 |
| Morphine | 27.0 |

Based on the ratio of naltrexone in an amount from about 0.5 to about 4 mg per 15 mg of hydrocodone, the approximate weight ratio of naltrexone to 1 mg of each opioid is set forth in Table 2.

TABLE 2
Weight Ratio of Naltrexone per Dose Opioid

| | Weight Ratio Naltrexone per |
|---|---|
| Opioid | 1 mg Opioid |
| Oxycodone | 0.037 to 0.296 |
| Codeine | 0.005 to 0.0944 |
| Hydrocodone | 0.033 to 0.267 |
| Hydromorphone | 0.148 to 1.185 |
| Levorphanol | 0.278 to 2.222 |
| Meperidine | 0.0037 to 0.09296 |
| Methadone | 0.056 to 0.444 |
| Morphine | 0.018 to 0.148 |

Based on the ratio of about 0.75 mg to about 3 mg naltrexone per 15 mg hydrocodone of naltrexone, the approximate weight ratio of naltrexone to 1 mg of each opioid is set forth in Table 3.

TABLE 3
Weight Ratio of Naltrexone per Dose Opioid

| Opioid | Weight Ratio Naltrexone |
|---|---|
| Oxycodone | 0.056 to 0.222 |
| Codeine | 0.0083 to 0.033 |
| Hydrocodone | 0.050 to 0.200 |
| Hydromorphone | 0.222 to 0.889 |
| Levorphanol | 0.417 to 1.667 |
| Meperidine | 0.0056 to 0.022 |
| Methadone | 0.083 to 0.333 |

Naloxone is an opioid antagonist which is almost void of agonist effects. Subcutaneous doses of up to 12 mg of naloxone produce no discernable subjective effects, and 24 mg naloxone causes only slight drowsiness. Small doses (0.4-0.8 mg) of naloxone given intramuscularly or intravenously in humans prevents or promptly reverses the effects of morphine-like opioid agonist. One mg of naloxone intravenously has been reported to completely block the effects of 25 mg of heroin. The effects of naloxone are seen almost immediately after intravenous administration. The drug is absorbed after oral administration, but has been reported to be metabolized into an inactive form rapidly in its first passage through the liver such that it has been reported to be only one fiftieth as potent as when parenterally administered. Oral dosage of more than 1 mg has been reported to be almost completely metabolized in less than 24 hours.

Other opioid antagonists, for example, cyclazocine and naltrexone, both of which have cyclopropylmethyl substitutions on the nitrogen, retain much of their efficacy by the oral route and their durations of action are much longer, approaching 24 hours after oral doses. A most preferred opioid antagonist is naltrexone. However, equiantagonistic oral doses of other opioid antagonists, including but not limited to naloxone, nalmephene, cyclazocine, and levallorphan can be utilized in accordance with the present invention. The ratio of such other antagonists to a particular pharmaceutical formulations can be readily determined without undue experimentation by one skilled in art who desires to utilize a different antagonist. Those skilled in the art may determine such ratios of other antagonists to the same or similar studies set forth in the examples herein. Thus, combinations of antagonists/pharmaceutical compositions which are orally administered in ratios which are equivalent to the ratio of, e.g., naltrexone to hydrocodone, set forth further herein, are considered to be within the scope of the present invention and within the scope of the appended claims. For example, in certain embodiments of the invention, naloxone is utilized as the opioid antagonist, the amount of naloxone included in the dosage form being large enough to provide an equiantagonistic effect as if naltrexone were included in the combination.

TABLE 4
Formulations Opioid Naltrexone*

| Opioid | Weight Ratio Naltrexone |
|---|---|
| SR Morphine | 15/2.25; 30/4.5; 60/9; 100/15; 20/3; 50/7.5; 100/15 |
| SR Oxycodone | 10/2 |
| Oxycontin | 20/4; 40/8; 80/16; 160/32 |
| Oramoeph, MS | 13/7.5 |
| Contin | |

*Amounts of narcotic are based on amounts used in presently available time-release narcotics.
*Naltrexone/morphine - 1:0.15
Oxycodin - 1:0.2
*Amount of antagonist may be arbitrarily higher or lower, depending on (a) desired effect; and (b) empirically determined availability in a particular chewed proportion.

In order to provide an antagonist which is only active upon chewing or crushing, the antagonist can for example be entrained in a matrix or coated with a substance that does not release under conditions found upon oral administration, e.g. not susceptible to gastric juices or conditions found in the intestinal track. Pills, granules, or the like can be coated with a pharmaceutically acceptable plastic or a composition like chitin or a hard wax. The coated antagonist would pass through the gut intact and undisolved. If, however, the granule was chewed or crushed, it would release the antagonist thus making it bioavailable in the gut. Likewise, a matrix which is insoluble in the gut, e.g. a plasticized matrix, would be made bioactive by chewing prior to administration. A wax which is insoluble at body temperature but melts at a higher temperature could also be incorporated as a deterrent to trying to melt the drug out or be used directly as the agent to prevent abuse or misuse.

It is understood that while the basic examples are limited to the opioids, one skilled in the art would be able to substitute other pharmaceutical compositions, agonists, and materials to coat the agonist based on the disclosure of the invention contained herein. Nothing in the language of the claims or interpretation thereof is intended to be limiting.

What is claimed is:

1. A controlled release, storage stable, pharmaceutical formulation intended for oral administration comprising: an orally active, therapeutic amount of a pharmaceutical composition in controlled release dosage form and an orally active antagonist to said pharmaceutical composition in sufficient quantity to counteract the effects of said pharmaceutical composition, said antagonist in a dosage form that is only orally effective as an antagonist if it is chewed or crushed before oral administration.

2. A pharmaceutical formulation according to claim 1 wherein said pharmaceutical composition is a controlled substance.

3. A pharmaceutical formulation according to claim 2 wherein said controlled substance is an opioid.

4. A pharmaceutical formulation according to claim 3 wherein said opioid is selected from a group comprising hydrocodone, hydromorphone, oxycodone, morphine sulfate, apomorphine, meperidone or a pharmacologically orally active salt thereof.

5. A pharmaceutical formulation according to claim 3 wherein said antagonist is selected from a group consisting of said pharmacologically orally active salts of naltrexone and naloxone.

6. A pharmaceutical formulation according to claim 1 where in said pharmaceutical composition is in a controlled release tablet dosage form.

7. A pharmaceutical formulation according to claim 1 wherein said pharmaceutical composition is in a controlled release capsule dosage form.

8. A pharmaceutical formulation according to claim 7 wherein said pharmaceutical composition is in said controlled release capsule dosage form comprising controlled release pills, granules or pellets.

9. A pharmaceutical formulation according to claim 1 wherein said antagonist is coated with a substance that will not release a sufficient quantity of said antagonist to counteract the effects of the pharmaceutical composition when taken orally but will release said sufficient amount if it is chewed or crushed before oral administration.

10. A pharmaceutical formulation according to claim 9 wherein said coating of said antagonist is selected from a group consisting of a pharmaceutically accepted plastic, chitin, or wax.

11. A method for reducing the potential harm or abuse possible from a controlled release oral dosage form of a pharmaceutical formulation caused by chewing or crushing said pharmaceutical formulation prior to oral administration comprising: combining a therapeutic amount of an orally active pharmaceutical composition in controlled release dosage form and an antagonist to said pharmaceutical composition in sufficient quantity to counteract the effects of said pharmaceutical composition when taken orally, said antagonist being in a dosage form that is only active orally if it is chewed or crushed before oral administration.

12. A method according to claim 11, wherein said pharmaceutical formulation is a controlled substance.

13. A method according to claim 12, wherein said controlled substance is an opioid.

14. A method according to claim 13, wherein said opioid is selected from a group consisting of the pharmacologically orally active salts of hydrocodone, hydromorphone, oxycodone, morphine sulfate, apomorphine, and meperidone.

15. A method according to claim 13 wherein said antagonist is selected from a group consisting of the pharmacologically orally active salts of naltrexone and naloxone.

16. A method according to claim 11 wherein said pharmaceutical composition and said antagonist are combined into an orally controlled release tablet dosage form.

17. A method according to claim 11 wherein said pharmaceutical composition and said antagonist are combined into an orally controlled release capsule dosage form.

18. A composition according to claim 17 wherein said pharmaceutical composition is in said controlled release capsule dosage form comprising controlled release pills, granules or pellets.

19. A method according to claim 11 wherein said antagonist is in the dosage form of a pill, granule or pellet which is coated with a substance that will not release an effective amount of said agonist when taken orally but will release an effective amount if said agonist is chewed or crushed before oral administration.

20. A method according to claim 19 wherein said coating on said antagonist is selected from a group consisting of a pharmacologically inactive plastic, chitin, or wax.
